(19) United States
(12) Patent Application Publication
(10) Pub. No.: US 2024/0050035 A1
(43) Pub. Date: Feb. 15, 2024

(54) VENOUS STRUCTURE IDENTIFICATION DEVICE AND DRESSING

(71) Applicant: 3M INNOVATIVE PROPERTIES COMPANY, (US)

(72) Inventors: Ganesh Prasad Kannan Durgam, Bangalore (IN); Rajalaxmi Dash, Bangalore (IN)

(21) Appl. No.: 18/267,630

(22) PCT Filed: Dec. 20, 2021
(86) PCT No.: PCT/IB2021/062047

§ 371 Date: Jun. 15, 2023

(30) Foreign Application Priority Data
Dec. 18, 2020 (IN) 202041055126

(51) Int. Cl. A61B 5/00; A61B 5/01

(52) U.S. Cl. CPC A61B 5/489; A61B 5/015; A61B 5/6833

(57) ABSTRACT

A thermal imaging device for locating a venous structure of the subject is disclosed. The device includes a substrate layer having a top and bottom surface and a plurality of sections of thermochromic elements positioned on the top surface. Each section of the thermochromic elements is separated from another section of the thermochromic elements. One or more sections of the thermochromic elements is sensitive to subjects skin temperature to indicate the venous structure of the subject.

### FIELD OF THE INVENTION

The present disclosure relates to a device that can be used in therapeutic and healthcare applications. In particular, the present disclosure pertains to a device having thermochromic elements that can indicate a change in temperature difference when placed on an object or subject that emits heat.

### BACKGROUND OF THE INVENTION

Patients may need to undergo numerous medical tests or procedures, and this involves collecting and analyzing blood samples and infusing fluid into patient's body. Such procedures need intravenous access or insertion of needle into the blood vessel of a patient's body. In many patients, these veins may be visible to naked eye, but for patients with dark skin or having more subcutaneous fat it may be difficult to identify the veins. Similarly, difficulty in identifying venous structure in old age patients, children and cancer patients who undergo chemotherapy, which causes collapse of veins, exists. So, multiple attempts may be made to insert the needle into the blood vessel, which is a painful and strenuous activity for patient and a healthcare professional (e.g. nurse). Multiple attempts to pierce the skin of patient or failed intravenous access attempts can lead to infection, seeping of blood into local tissues (i.e. a condition of Hematoma), infiltration of medication to surrounding tissues, Phlebitis or inflammation of vein, air embolism (i.e., condition of air entering the vein through intravenous (IV) tubing or improper syringe injection), punctured vein, nerve damage (e.g. damage of sensory nerve proximate to the vein), Diaphoresis (patient sweating due to fear), Syncope (patient fainting due to fear) and so on.

Another common procedure used to locate vein is application of a tourniquet on patient's arm. Normally the tourniquet is placed few inches away from the site and is used to apply pressure by slowing the venous flow in superficial veins without affecting the arterial flow. This causes blood to accumulate in the veins and distending them making it easier to locate the vein. The veins respond to pressure difference created and they enlarge so that it is visible, or it can be felt by touching the patient's arm. This technique is not always successful, for instance, if the patient is an old aged person or a child it may be difficult to identify and if the veins are dark, they may not be visible through a dark skin.

Other numerous imaging techniques to simpler techniques like simple red light transillumination developed and available to identify venous structure of the humans and animals An example is a miniature Magnetic Resonance Imaging (MRI) device that uses an imaging technique to see small structures such as tiny ligaments and nerves in hand of a user. Even though such techniques are available, they are usually complex because of a need for more than one person to handle multiple steps or procedures for identification and cannulation in the patient's arm, and thus expensive.

Therefore, there is a need for a simple to use and effective method for identifying the presence of blood vessels.

### SUMMARY OF THE INVENTION

The present disclosure provides a thermal imaging device for locating a venous structure in a subject. The thermal imaging device has a simple to use design that can be removably placed on the skin of the subject and used to locate the venous structure for inserting a venous access device into the venous structure. In further embodiments, once venous catheterization is completed, the user can remove the device conveniently and another dressing can be secured at the catheterization site.

In an embodiment, a device for locating a venous structure of the subject is disclosed. The device includes a substrate layer having a top and bottom surface and a plurality of sections of thermochromic elements positioned on the top surface. Each section of the thermochromic elements is separated from another section of the thermochromic elements. One or more sections of the thermochromic elements is sensitive to subject's skin temperature to indicate the venous structure of the subject.

In certain embodiments, the device is a thermal imaging dressing and includes a substrate layer, a plurality of sections of thermochromic elements positioned on a top surface of the substrate layer, and an adhesive backing layer removably disposed on a portion of the top surface of the substrate layer. The adhesive backing layer can be removed to place the thermal imaging dressing on the skin. The plurality of sections of thermochromic elements are arranged such that each section of the thermochromic elements is separated from another section and are sensitive to subject's skin temperature to indicate a venous structure of the subject.

In an embodiment, the substrate layer of the thermal imaging dressing and device, include a tear-off tab for peeling the substrate layer from the subject's skin and a plurality of perforations that facilitates the substrate layer to be peeled off from the subject's skin. These perforations help the substrate layer to be peeled off conveniently once the venous access device is secured into the venous structure of the subject.

In another embodiment, a method for locating a blood vessel on subject's body uses the thermal imaging device. The method includes providing a substrate layer having a plurality of sections of thermochromic elements positioned on a top surface of the substrate layer. Each section of the thermochromic elements is separated from another section of the thermochromic elements. The one or more sections of the thermochromic elements is sensitive to subject's skin temperature to indicate the blood vessel. The method further includes placing the substrate layer on subject's skin for locating the blood vessel and inserting a venous access device into a desired position of the blood vessel that is indicated by the substrate layer.

### BRIEF DESCRIPTION OF DRAWINGS

The proposed thermal imaging device and thermal imaging dressing for locating a venous structure of a subject and method thereof are illustrated in the accompanying drawings, throughout which like reference letters indicate corresponding parts in the various figures. The embodiments herein will be better understood from the following description with reference to the drawings, in which:

FIG. 1A illustrates a perspective view of a device for locating a venous structure of the subject, according to an embodiment of the disclosure;

FIG. 1B illustrates a cross-sectional view of the device of FIG. 1A along lines 2-2;

FIG. 2A illustrates a device for locating a venous structure of the subject according to another exemplary embodiment;

FIG. 2B illustrates the device of FIG. 2A along lines 3-3;

FIG. 3 schematically illustrates multiple such units arranged in an isolated manner according to an exemplary embodiment;

FIG. 4 schematically illustrates different layers of a unit of thermochromic elements such as according to an embodiment;

FIG. 5A and 5B illustrate a device having thermochromic elements for detecting the venous structure of a subject according to an exemplary embodiment;

FIG. 6A illustrates a perspective view of a thermal imaging dressing for locating a venous structure of the subject according to an exemplary embodiment;

FIG. 6B illustrates a cross-sectional view of the thermal imaging dressing of FIG. 6A;

FIG. 7 illustrates a perspective view of a thermal imaging dressing showing a venous structure according to an embodiment; and

FIG. 8 illustrates a thermal imaging dressing having a substrate layer according to another exemplary embodiment.

### DETAILED DESCRIPTION OF THE DRAWINGS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

The present disclosure is directed to a thermal imaging device for locating a venous structure of the subject is disclosed. The device includes a substrate layer having a top and bottom surface and a plurality of sections of thermochromic elements positioned on the top surface. Each section of the thermochromic elements is separated from another section of the thermochromic elements. One or more sections of the thermochromic elements is sensitive to subject's skin temperature to indicate the venous structure of the subject.

In some embodiments, the device is a thermal imaging dressing include a substrate layer, a plurality of sections of thermochromic elements positioned on a top surface of the substrate layer, and an adhesive backing layer removably disposed on a portion of the top surface of the substrate layer. The adhesive backing layer can be removed to place the thermal imaging dressing on the skin. The plurality of sections of thermochromic elements are arranged such that each section of the thermochromic elements is separated from another section and are sensitive to subject's skin temperature to indicate a venous structure of the subject.

The device can be removably placed on the skin of the subject and used to conveniently locate the venous structure for inserting a venous access device into the venous structure.

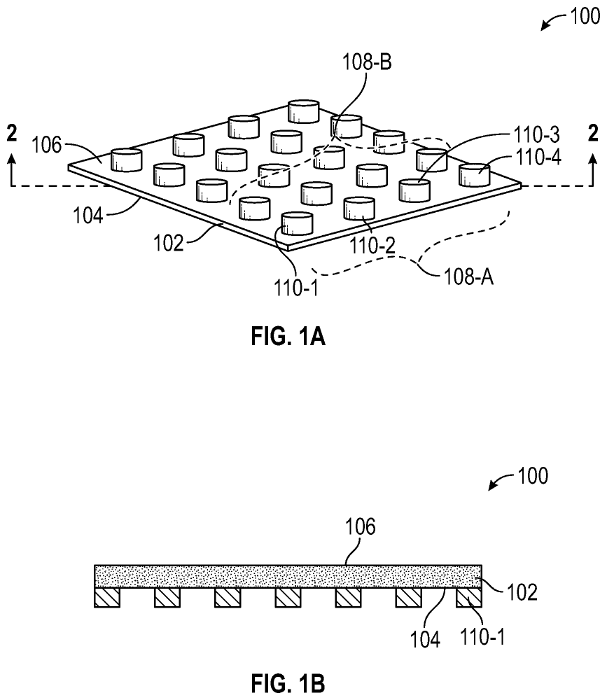

Referring now to the figures and more particularly to FIGs. 1A and 1B showing a device 100 for locating a venous structure of the subject according to an embodiment. FIG. 1A shows a perspective view and FIG. 1B shows a cross-sectional view of the device 100 taken along the lines 2-2. The device 100 includes a substrate layer 102 having a top surface 104 and a bottom surface 106. The substrate layer may be composed of a low heat conductive material, a thermally neutral material and so on. The substrate layer 102 may be composed of a polymer material including natural or synthetic polymers. In an embodiment, the substrate layer 102 may be configured in a form of thin film structure. A plurality of sections of thermochromic elements 108 are positioned on the bottom surface 106 of the substrate layer 102. The thermochromic elements 108 are sensitive to subject's skin temperature ranges and capable of indicating a venous structure (or vein) of the subject. The thermochromic elements 108 are sensitive to temperature and change color with exposure to heat. The thermochromic elements 108 may be receptive to temperatures within the range of 32-40 degree Celsius which includes the normal human body temperature (i.e. 37 degree Celsius or 98.6 degree Fahrenheit). The thermochromic elements may be capable of or configured to respond to small change in temperature. In an embodiment, the thermochromic elements 108 may be capable of showing different colors to indicate different temperature ranges.

The thermochromic elements may be in the form of thermochromic crystals. In an embodiment, the thermochromic elements 108 may be composed of an encapsulated enantiotropic cholesteric liquid crystalline phase material exhibiting a mesophase color change at temperature corresponding to human skin temperatures. The thermochromic materials are generally organic (i.e. carbon-based) leuco-dye mixtures and compose color developer, color former (e.g. an ester that determines the base color) and a solvent. The leuco-dye mixtures may start as transparent or have a color and changes color once the temperature changes. Molecules in leuco-dye mixtures shift back and forth between two structures known as leuco form (i.e. colorless) and non-leuco form (i.e. colored). These two forms absorb and reflect light in different manner thereby they appear to display different colors. Other thermochromic materials used may include liquid crystals. The liquid crystals can be in form of multiple different phases such as, nematic and smectic. Some liquid crystals start get excited at low temperatures to enter smectic phase in which molecules in the liquid crystals forms layers that slide past one another. Then at high temperature, the liquid crystals shift to a different phase such as chiral or cholesteric and may start to display shifting colors as they get hotter. When the temperature reaches certain high level, the molecules stop behaving like liquid crystals and enter an isotropic state by showing same optical properties in every direction, thus may be transparent once again.

Each section of the thermochromic elements positioned on the substrate layer is separated from another section of the thermochromic elements. For example, a section of thermochromic elements 108-A may be separated from another section of thermochromic elements 108-B. Similarly, other sections of thermochromic elements are separated from each other as shown in FIG. 1B. Each section includes multiple thermochromic elements arranged in a particular manner In an embodiment, the section of thermochromic elements 108-A may include multiple thermochromic elements (such as, thermochromic elements 110-1, 110-2, 110-3, and 110-4) arranged in a straight row configuration (i.e. a linear manner). The thermochromic element 110-1 may be separated from an adjacent thermochromic element 110-2 by a distance. Thus, multiple rows of thermochromic elements form different sections for example, sections of thermochromic elements 108-A and 108-B. Even though each section of thermochromic elements are arranged in straight row configuration, these sections of thermochromic elements can be arranged in any other manner on the substrate layer 102 to ensure isolation between the sections, according to various other embodiments. Further, the thermochromic element is depicted in FIG. 1A in a cylindrical form according to an embodiment, however it may be noted that the thermochromic elements can have any other shape such as, cuboidal, cube, truncated spherical, truncated conical (circular or elliptical), cylindrical (circular or elliptical), triangular or pentagonal prism, hexagonal prism and their derivatives thereof, and different sizes according to various other embodiments.

When the device 100 is placed on subject's skin, the thermochromic elements contact the skin to get excited and change color, as they are sensitive to body heat. The thermochromic elements detect a temperature difference between the vein and surrounding skin and shows one color along the vein. After passage of time, and if the thermochromic elements are closely placed to each other, the color quickly diffuses due to transfer of heat or temperature to adjacent thermochromic elements. In other words, the heat experienced by few thermochromic elements in contact with the vein laterally transfers to adjacent thermochromic elements to diffuse the color. However, in the device 100, as each section of thermochromic elements is separated or isolated from another section, the lateral radiation of heat reduces thereby the color change indicating the vein stays for a prolonged time. Therefore, each thermochromic element (110-1 or 110-2) act as an isolated sensor not affected by heat radiation from adjacent thermochromic element.

The device 100 having the thermochromic elements may be formed in the shape of circles, rectangles, squares, triangles, octagons and so on, and at suitable size so that it can be conveniently placed or positioned on the subject's skin for locating the venous structure.

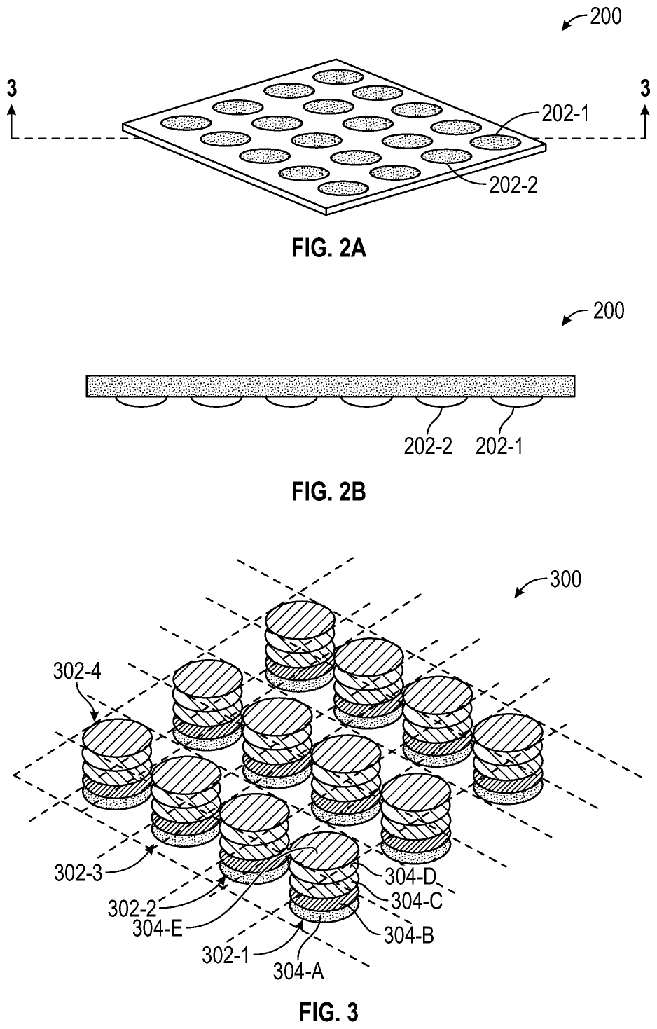

FIG. 2A and 2B illustrate a device 200 for locating a venous structure of the subject according to another exemplary embodiment. FIG. 2A shows a perspective view and FIG. 2B shows a cross-sectional view of the device 200 taken along the lines 3-3. The device 200 have thermochromic elements (e.g. thermochromic elements 202-1 and 202-2) positioned on its bottom surface of a substrate layer 204. The substrate layer 204 is configured to hold the thermochromic elements in place. Each thermochromic element act as an isolated sensor because the thermochromic elements are separated from each other at a distance. The thermochromic elements have a dot configuration or a circular configuration. In an embodiment, the thermochromic element may have a micro-dot structure. Examples of other shapes of the thermochromic elements, may be elliptical, square, rectangle, triangle, parallelogram and so on. As shown in FIG. 2B, the thermochromic elements may have an inverted curved shape where the peak surface of the thermochromic elements touch the subject's skin.

The device 200 when positioned on the subject's body, the peak of thermochromic elements touches the subject body and gets excited by body heat. The thermochromic elements that are in contact with skin proximal to vein changes color due to the heat transferred from the vein to them. This color change helps a user (e.g. a healthcare professional) to locate the vein and insert a venous access device into the vein.

Thermochromic elements may be structured or arranged in the form of multiple units. A unit of thermochromic elements may have a peak surface that can contact the subject's skin. In an embodiment each unit of thermochromic elements may have a column structure. Each unit of thermochromic elements may have different shapes, but not limited to, conical, cylindrical, cuboidal, spherical, prism, square and triangular pyramid, and any three-dimensional shape. FIG. 3 schematically shows multiple such units arranged in an isolated manner according to an exemplary embodiment. The units such as, a unit 302-1, a unit 302-2, a unit 302-3 and a unit 302-4 form a section of thermochromic elements. All units of thermochromic elements are positioned on a substrate layer even though it is not illustrated in FIG. 3. In an embodiment, each unit of thermochromic elements may have multiple layers of thermochromic elements arranged to form a column structure. A layer may include thermochromic crystals or molecules and all the layers form a chain of thermochromic crystals or molecules. For example, the unit 302-1 have multiple layers such as, 304-A, 304-B, 304-C, 304-D and 304-E. The unit of thermochromic elements may be formed (for example, printed) on the substrate layer in the form of a patterned well using a technique for example, microreplication technique. In an example, in the unit of thermochromic elements 302-1, a micro-replicated dot represents the layer 304-A placed on the substrate layer. Another layer 304-B (i.e. another micro-replicated dot) may be overlaid on the layer 304-A. Similarly, other layers 304-C, 304-D and 304-E are overlaid or stacked on top of each other thereby forming the unit 302-1 having the column structure or patterned well structure. Other units of thermochromic elements 302-2, 302-3, and 302-4 have similar layered structure and can be constructed using the microreplication technique. For forming units of thermochromic elements using the microreplication technique, for instance a first layer of thermochromic element (i.e. micro-replicated dots) for each unit may be overlaid on the substrate layer. Thereafter other layers such as second, third, fourth and fifth layers of each thermochromic element unit are overlaid on top of another to form all the thermochromic element units. In another embodiment, each unit of thermochromic elements may be constructed as a single column of thermochromic elements without having multiple layers using a micro-replication technique. However, it should be borne in mind that other techniques apart from micro-replication or printing technique for forming units of thermochromic elements are also contemplated by the present disclosure.

A unit of thermochromic elements is positioned or placed away from another unit at a distance or with a small gap around the unit. Taking an example, temperature experienced at the unit 302-1 may not affect the adjacent unit 302-2 as they are spaced apart from each other. In other words, the unit 302-1 will react to a temperature experienced by it and will not be influenced by temperature experienced by the adjacent unit 302-2. As a result, the unit 302-1 act as an isolated sensor of heat and is more likely to retain the color indication for a longer duration without being affected by heat radiation from the units 302-2 or 302-3.

In an embodiment each unit of thermochromic elements may have an inverted structure for example, an inverted curve or inverted cone or inverted cylinder structure with the peak surface of the inverted structure contacting the skin. Considering the unit 302-1, a peak surface of the unit 302-1 and more particularly the peak on the layer 304-A touch the skin when the device is placed on the subject's skin. The temperature experienced in the layer 304-A is radiated to other layers 304-B, 304-C, 304-D and 304-E so that whole unit 302-1 gets excited by heat and emits a color indicating the temperature. Multiple such units get excited by the heat and emits a color to indicate the location of vein from the skin.

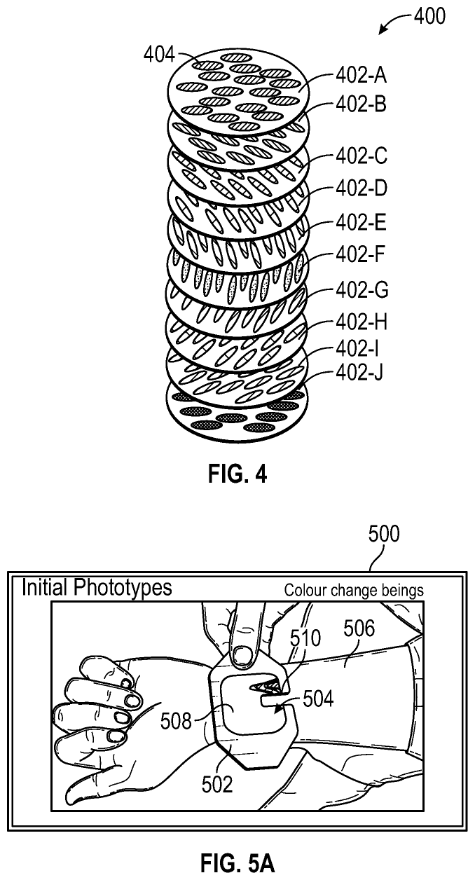

FIG. 4 is schematic illustration of different layers of a unit of thermochromic elements such as, a unit 400 according to an embodiment. The unit 400 includes multiple layers of thermochromic elements for example, 10 layers. The layers 402-A, 402-B, 402-C, 402-D, 402-E, 402-F, 402-G, 402-H, 402-I and 402-J. These layers are overlaid one top of another as shown. For example, each layer may be overlaid on top of another layer of thermochromic elements as explained earlier in conjunction with FIG. 3. Each layer includes multiple molecules for example, cellulose nanorods. For example, the layer 402-A includes many molecules such as, a molecule 404. The molecules are customized chiral organic molecules. The molecules will be arranged in a particular manner in each layer. In an embodiment, the arrangement of molecules in a layer (e.g. the layer 402-A) may be different from another layer (e.g. the layer 402-B). When the unit 400 comes in contact with heat or receives heat, the molecules in each layer rotate and align themselves at a particular angle to each other. Based on this angle of alignment they reflect and exhibit a corresponding color. The molecules are reversible in nature, which means when the temperature goes down or if the unit 400 is no longer receiving heat, the molecules return to original alignment and thus the color is not exhibited.

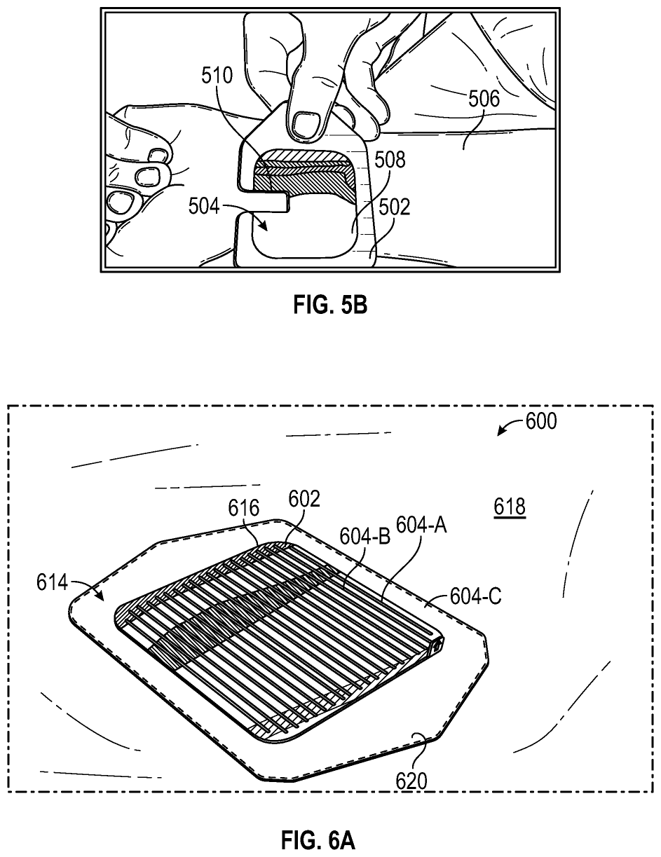

Now moving to FIG. 5A which illustrates a device 500 having thermochromic elements for detecting the venous structure of a subject according to an exemplary embodiment. The device 500 comprises a frame structure 502 for holding a substrate layer 504 (similar to the substrate layer 102 or the substrate layer 304) having the thermochromic elements. In an embodiment, the substrate layer 504 may be made of a polymer material having a black background. The frame structure 502 provides adequate support to the substrate layer so that it can be in place and is well protected. The frame structure 502 includes an open area 503 (or a hollow slot) in the middle that enables the substrate layer 504 to be exposed. In an embodiment, the substrate layer 504 may be slid into a slit opening provided in the frame structure 502. The device 502 may be placed or positioned on hand 506 of the subject so that the substrate layer 504 gets in touch with the skin. The thermochromic elements on the lower surface of the substrate layer 504 touch the skin and gets excited or activated due to the heat from a venous structure in the hand The thermochromic elements exhibit some color or changes color (i.e. color indication 507) to indicate the location of the venous structure in the hand, as can be seen on a surface 508 of the substrate layer 504. A user such as, a healthcare professional can use this color indication and insert a venous access device into the venous structure. In an embodiment, the device 500 may include a slot 510 that guides the user to insert the venous access device. The device 500 can be moved around the hand 506 to center or align the slot 510 to the vein so that user can insert the venous access device in the vein, as shown in FIGS. 5A and 5B. The vein location is depicted by a color indication 512 in FIG. 5B. Thereafter, the device 500 can be removed. A suitable dressing can be placed over the inserted venous access device to secure the venous access device in position.

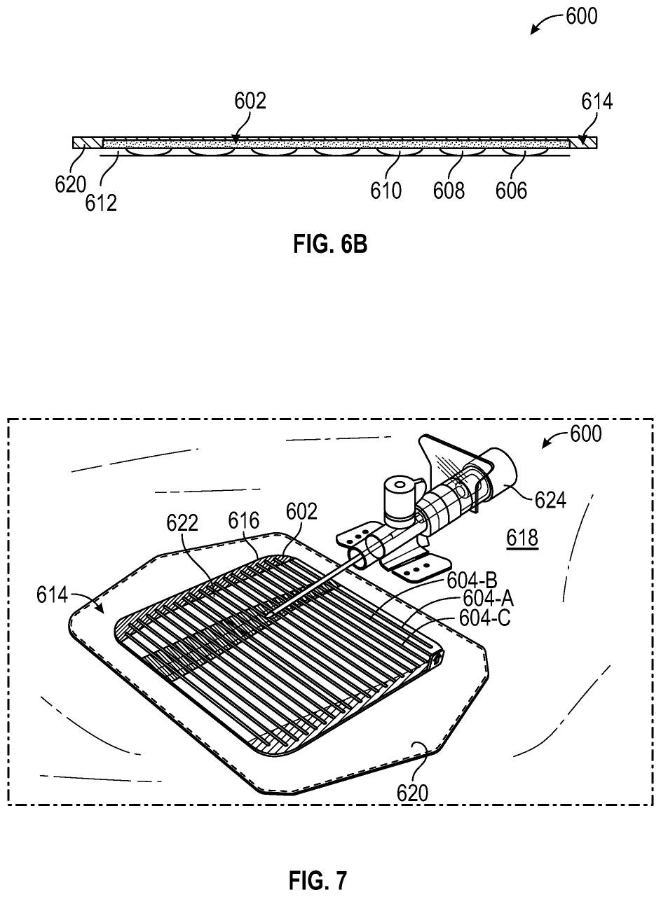

The thermal imaging device can be a dressing with the substrate layer along with the thermochromic elements positioned on it. FIG. 6A and 6B illustrate a thermal imaging dressing 600 for locating a venous structure of the subject according to another exemplary embodiment. FIG. 6A shows a perspective view and FIG. 6B shows a cross-sectional view of the thermal imaging dressing 600. The thermal imaging dressing 600 includes a substrate layer 602 having a plurality of sections of thermochromic elements for example, a section 604-A, a section 604-B and 604-C. Each section of thermochromic elements may be formed by multiple units of thermochromic elements. For instance, the section 604-A includes multiple units of thermochromic elements like a unit 606 when seen along section line 4-4 as shown in FIG. 6B. Similarly, the section 604-B and 604-C include numerous units of thermochromic elements like a unit 608 and a unit 610 respectively. An adhesive backing layer 612 is removably disposed on a bottom surface of the substrate layer 602. The adhesive backing layer 612 may be composed of a repositionable adhesive, a pressure sensitive adhesive, and so on. The adhesive backing layer 612 covers the thermochromic elements or overlaid on the thermochromic elements. The substrate layer 602 and the thermochromic elements are held or supported by a frame structure 614. The frame structure 614 defines an open area of the substrate layer 602. As shown in FIG. 6A, the frame structure 614 may have an opening 616 that allows the substrate layer 602 with the thermochromic elements to be exposed out of the frame structure 614. The open area refers to a portion of the substrate layer 602 and the thermochromic elements that is exposed. In an embodiment, the substrate layer 602 may be a thin or flexible film and the frame structure 614 may cover along the edges of the substrate layer 602 and thereby give enough strength to form the thermal imaging dressing 600.

Now for applying on the subject's skin e.g. skin 618, the adhesive backing layer 612 is removed and the thermal imaging dressing 600 is placed on the skin. In an embodiment, in order to securely position the thermal imaging dressing 600 a repositionable adhesive layer may be provided along an edge portion 620 of the frame structure 614. The edge portion 620 is the portion around the opening 616. The repositionable adhesive layer may be provided so that the dressing 600 can be removed after usage. When the thermochromic elements contact the skin 618, heat from the venous structure underlying the skin 618 excites the thermochromic elements and thereby a color change can be observed. The thermochromic elements that receive heat from the venous structure exhibit the color change rather than any of the surrounding thermochromic elements which are away from the venous structure. For instance, few sections of thermochromic elements (such as, the sections 604-B and 604-C) may be proximate to the venous structure or is in contact with skin area that is close to the venous structure and thus show a color change whereas the section 604-A may not be close to the venous structure and may not show a color change or show a different color. More specifically, thermochromic elements in the section 604-A may not experience heat and thus may not be excited or may get heat at different rate or temperature as compared to the thermochromic elements in other sections 604-B and 604-C, and hence exhibit different color.

In an embodiment, the thermochromic elements may be capable of changing or exhibiting different color based on different temperature ranges. The color of the thermochromic elements may vary from a non-reflective black color through spectral colors and then to black color again. Generally, at high temperature the thermochromic elements will reflect a blue-violet color and while in low temperature it will reflect a red-orange color.

In other instance, only few units in each section of the thermochromic elements (e.g. sections 604-A, 604-B, 604-C and 604-N) may get excited and exhibit a color change showing a portion 622 of venous structure as shown in FIG. 7. The other units of thermochromic elements away from the venous structure neither get excited nor exhibit any color change as shown in FIG. 7. As a major portion of the venous structure (i.e. the portion 622) is visible to the user (e.g. the healthcare professional), the user can insert a venous access device 624 into the portion 622 of venous structure. The venous access device 624 can pierce through the substrate layer 602 to gain access into the venous structure.

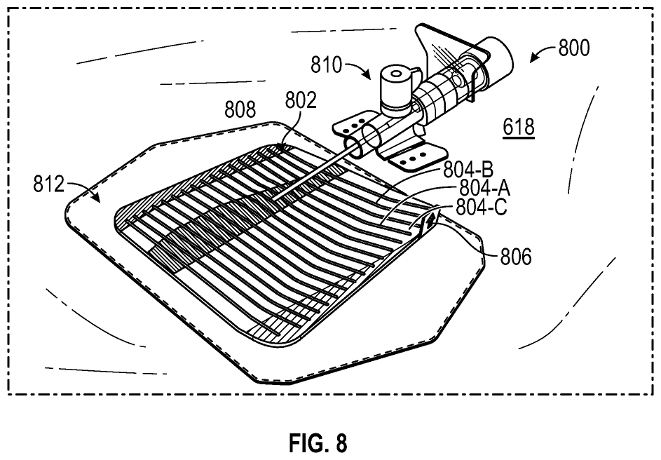

Moving to FIG. 8, a thermal imaging dressing 800 having a substrate layer 802 is shown according to another exemplary embodiment. The substrate layer 802 may include an area of weakness that enable the layer 802 to be removed conveniently from the skin. The area of weakness may be in the form series of perforations, slits, cuts or the like. In an embodiment, a plurality of perforations such as perforations 804-A, 804-B and 804-C is provided on the substrate layer 802. The perforation may be in the form of a dot, hole, straight line cut, linear slit, a sinusoidal wave slit, V-shaped cut or Y-shaped cut or a zig-zag cut on the substrate layer 802. In an embodiment, the perforations are positioned apart from each other. For example, the perforations 804-A, 804-B and 804-C are spaced apart from each other as shown in FIG. 8. The substrate layer 802 have a tear-off tab 806 that can be used to remove the substrate layer 802 from the skin. The tear-off tab 806 may be provided at any corner or edges of the substrate layer 802. When the tear-off tab 806 is pulled by the user the substrate layer 802 tears or breaks through the perforations so that substrate layer 802 can be easily peeled off from the skin. For example, tearing of the substrate layer 802 along the perforation 804-A can be seen in FIG. 8.

During application, the thermal imaging dressing 800 is placed on the skin after removing the adhesive backing layer (e.g. the adhesive backing layer 610). The thermal imaging dressing indicates a portion 808 of the venous structure by showing a change in color. A venous access device 810 can be inserted into a desired portion i.e., the portion 808 through the substrate layer 802. The user can pull the tear-off tab 806 to remove the substrate layer 802. The perforations in the substrate layer 802 enable it to break or tear around the venous access device 810 and helps in removing the substrate layer 802 conveniently. A frame structure 812 of the thermal imaging dressing 800 can be removed easily due to the presence of repositionable adhesive layer around the edge portion if the frame structure 812.

The thermal imaging dressing, such as the dressing 800 can be used as any another normal dressing that can be lightly placed on the hand to reveal the vein. Further, as the thermochromic elements are separated or spaced apart from each other in the dressing, the color change exhibited by the thermochromic elements last for a longer duration through the substrate layer thereby providing the healthcare person more time for performing cannulation. Moreover, the dressing also helps to reduce the number of venous punctures, be used for different types of patients, and avoid any pain for the patient. The dressing is also lightweight that makes it simple to use for a user and allows identification of vein accurately.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

Following are the reference numerals:

| Numeral | Description |
|---|---|
| Device | 100, 200, 500 |
| Substrate Layer | 102 |
| Top Surface of Substrate layer | 104 |
| Bottom Surface of Substrate Layer | 106 |
| Thermochromic element | 108 |
| Section of thermochromic elements | 110-1 to 110-4 |
| Thermochromic elements | 202-1 and 202-2 |
| Substrate layer | 204 |
| Thermochromic Units | 302-1 to 302-4 |
| Layers of thermochromic elements | 304-A to 304-E |
| Unit of thermochromic elements | 400 |
| Layers of Unit 400 | 402-A to 402-J |
| Frame structure | 502 |
| Substrate layer | 504 |
| Open Area | 503 |
| Hand | 506 |
| Surface of substrate layer 504 | 508 |
| Slot | 510 |
| Color indication | 512 |
| Thermal Imaging Dressing | 600 |
| Substrate layer | 602 |
| Sections of thermochromic elements | 604-A to 604-C |
| Units of thermochromic elements | 606, 608, 610 |
| Adhesive backing layer | 612 |
| Frame structure | 614 |
| Opening | 616 |
| Skin | 618 |
| Edge portion | 620 |
| Portion of venous structure | 622 |
| Venous access device | 624 |
| Dressing | 800 |
| Substrate layer | 802 |
| Perforations | 804-A to 804-C |
| Tear-off tab | 806 |
| Portion in the substrate layer 802 | 808 |
| Venous access device | 810 |
| Frame structure | 812 |

1. A device for locating a venous structure of a subject, the device comprising:
a substrate layer having a top and bottom surface; and
a plurality of sections of thermochromic elements positioned on the top surface of the substrate layer, wherein each section of the thermochromic elements is separated from another section of the thermochromic elements.

2. The device of claim 1, wherein the one or more sections of the thermochromic elements shows a change in color to locate the venous structure.

3. The device of claim 1, wherein a section of the thermochromic elements comprises a plurality of units of thermochromic elements on the substrate layer.

4. The device of claim 3, wherein the section of thermochromic elements is disposed in a linear manner on the substrate layer.

5. The device of claim 3, wherein a unit of thermochromic element have a peak surface contacting the subject's skin.

6. The device of claim 3, wherein a unit of thermochromic elements is isolated from another unit of thermochromic element.

7. The device of claim 6, wherein the unit of the thermochromic elements have a micro-replicated dot structure.

8. The device of claim 1, wherein the substrate layer is a low heat conductive layer.

9. The device of claim 1 further comprising a frame structure for holding the substrate layer, wherein the frame structure defines an open area of the substrate layer that allows contact with subject's skin.

10. The device of claim 1, wherein the substrate layer comprises a plurality of perforations that facilitate the substrate layer to be peeled off from the subject's skin subsequent to securing a venous access device to the venous structure of the subject.

11. A thermal imaging dressing for locating a venous structure of a subject, the thermal imaging dressing comprising:
a substrate layer having a top and bottom surface;
a plurality of sections of thermochromic elements positioned on the top surface of the substrate layer, wherein each section of the thermochromic elements is separated from another section of the thermochromic elements; and
an adhesive backing layer removably disposed on the top surface of the substrate layer.

12. The thermal imaging dressing of claim 11, wherein a section of the thermochromic elements comprises a plurality of units of thermochromic elements on the substrate layer.

13. The thermal imaging dressing of claim 12, wherein the section of thermochromic elements is disposed in a linear manner on the substrate layer.

14. The thermal imaging dressing of claim 12, wherein a unit of thermochromic element is isolated from another unit of thermochromic element.

15. The thermal imaging dressing of claim 14, wherein unit of the thermochromic elements have a micro-replicated dot structure.

16. The thermal imaging dressing of claim 11, wherein the substrate layer is a low heat conductive layer.

17. The thermal imaging dressing of claim 11 further comprising a frame structure for holding the substrate layer, wherein the frame structure defines an open area of the substrate layer that allows contact with subject's skin.

18. The thermal imaging dressing of claim 11, wherein the substrate layer comprises:
a tear-off tab for peeling the substrate layer from the subject's skin; and
a plurality of perforations that facilitates the substrate layer to be peeled off from the subject's skin subsequent to securing venous access device to the venous structure of the subject.

19. A method for locating a blood vessel on a subject's body, the method comprising:
providing a substrate layer having a plurality of sections of thermochromic elements positioned on a top surface of the substrate layer, wherein each section of the thermochromic elements is separated from another section of the thermochromic elements, the one or more sections of the thermochromic element is sensitive to subject's skin temperature to indicate the blood vessel;
placing the substrate layer on subject's skin for locating the blood vessel; and
inserting a venous access device into a desired position of the blood vessel that is indicated by the substrate layer.

20. The method of claim 19 further comprising removing the substrate layer from the subject's skin after the venous access device is inserted into the blood vessel, wherein the substrate layer comprises a plurality of perforations that facilitates removal of the substrate layer.

21. (canceled)
